(19) United States
(12) Patent Application Publication
(10) Pub. No.: US 2021/0330564 A1
(43) Pub. Date: Oct. 28, 2021

(54) AQUEOUS ORAL CARE COMPOSITIONS

(71) Applicant: Mahnaz Rashti, Beverly Hills, CA (US)

(72) Inventor: Mahnaz Rashti, Beverly Hills, CA (US)

(21) Appl. No.: 16/858,969

(22) Filed: Apr. 27, 2020

(51) Int. Cl. A61K 8/34; A61K 8/36; A61K 8/60; A61K 8/20; A61Q 11/00; A61K 8/9789

(52) U.S. Cl. CPC A61K 8/345; A61K 8/361; A61K 8/60; A61K 2800/59; A61Q 11/00; A61K 8/9789; A61K 2800/30; A61K 8/20

(57) ABSTRACT

Disclosed herein are aqueous oral care compositions and methods of making such compositions. One aspect of the aqueous oral care composition comprises sodium bicarbonate, glycerin, sodium gluconate, erythritol, magnesium chloride, zinc citrate, and potassium citrate. Additional aspects include methods for making an aqueous oral care composition.

### BACKGROUND OF THE INVENTION

Aqueous oral care compositions (mouthwashes) are used to i) promote tooth and gum health, ii) prevent halitosis, iii) reduce plaque and tartar accumulation, iv) promote a healthy oral microbiome, and v) provide antimicrobial and anti-inflammatory properties. Most commercially available mouthwashes are characterized by an acidic pH and the presence of alcohol and/or chemicals that should not be ingested.

While the acidity of mouthwash is used for preservation and stable shelf life, it can actually cause enamel erosion. Mouthwashes with alcohol may have antimicrobial properties, but they can also irritate and burn oral tissue and cause dry mouth, which can lead to tooth sensitivity, bad breath, and even cavities. Furthermore, if specific individuals ingest alcohol (e.g., babies and children), there can be detrimental effects to their health and safety.

It would be advantageous to have oral care compositions having antimicrobial, anti-inflammatory, and anti-cavity properties without the adverse effects of acidifying preservatives and alcohol.

### SUMMARY OF THE INVENTION

Disclosed herein are aqueous oral care compositions and methods of making such compositions.

One aspect of the invention is an aqueous oral care composition comprising: sodium bicarbonate; glycerin; sodium gluconate; erythritol; magnesium chloride; zinc citrate; and potassium citrate. In a further aspect, the aqueous oral care composition further comprises at least one extract. In yet another aspect, the at least one extract is selected from the group of consisting of a flavor extract, an antimicrobial extract, an anti-inflammatory extract, an antioxidant extract, an anti-cavity extract, an antibacterial extract, an antiviral extract, an anti-inflammatory extract, a digestive extract, and an anti-allergy extract. In yet a further aspect, the at least one extract is selected from the group consisting of a pomegranate extract, a thyme extract, a licorice extract, a turmeric extract, and a peppermint extract. In another aspect, the aqueous oral care composition has a pH in the range of 7 to 9. In yet another aspect, the aqueous oral care composition does not contain alcohol and is ingestible. In another aspect, aqueous oral care composition can be safely stored at room or ambient temperature for a usefully long shelf life. In a further aspect, the aqueous oral care composition can be safely stored at room or ambient temperature for at least 1 year. In another aspect, the aqueous oral care composition formulation further comprises water.

One aspect of the invention is an aqueous oral care composition formulation that comprises: a percentage of sodium bicarbonate between about 0.95% w/w and about 1.05% w/w of the formulation; a percentage of glycerin between about 28.5% w/w and about 31.5% w/w of the formulation; a percentage of sodium gluconate between about 0.38% w/w and about 0.42% w/w of the formulation; a percentage of erythritol between about 3.8% w/w and about 4.2% w/w of the formulation; a percentage of magnesium chloride between about 0.475% w/w and about 0.525% w/w of the formulation; a percentage of zinc citrate between about 0.19% w/w and about 0.21% w/w of the formulation; and a percentage of potassium citrate between about 1.425% w/w and about 1.575% w/w of the formulation. In a further aspect, the aqueous oral care composition formulation further comprises: pomegranate extract, wherein a percentage of pomegranate extract is between about 0.095% w/w and about 0.105% w/w of the formulation; thyme extract, wherein a percentage of thyme extract is between about 0.095% w/w and about 0.105% w/w of the formulation; licorice extract, wherein a percentage of licorice extract is between about 0.095% w/w and about 0.105% w/w of the formulation, and; turmeric extract, wherein a percentage of turmeric extract is between about 0.095% w/w and about 0.105% w/w of the formulation. In yet a further aspect, the aqueous oral care composition formulation further comprises: peppermint flavor extract, wherein a percentage of peppermint flavor extract is between about 0.19% w/w and about 0.21% w/w of the formulation. In yet a further aspect, the aqueous oral care composition formulation further comprises: water, wherein a percentage of water is about 57% w/w and about 63% w/w of the formulation. In this aspect, the aqueous oral care composition does not contain alcohol and is ingestible.

One aspect of the invention is a method for producing an aqueous oral care composition comprising the steps of: adding water to a vessel; adding zinc citrate and potassium citrate to the vessel; mixing the water, zinc citrate, and potassium citrate; adding a plurality of additional ingredients to the vessel comprising sodium bicarbonate, glycerin, sodium gluconate, erythritol, magnesium chloride, and at least one extract; and mixing the ingredients to produce a solution. In another aspect, the at least one extract is selected from the group of consisting of a flavor extract, an antimicrobial extract, an anti-inflammatory extract, an antioxidant extract, an anti-cavity extract, an antibacterial extract, an antiviral extract, an anti-inflammatory extract, a digestive extract, and an anti-allergy extract. In a further aspect, the at least one extract is selected from the group consisting of a pomegranate extract, a thyme extract, a licorice extract, a turmeric extract, and a peppermint extract. In another aspect of the method, the solution comprises: a percentage of sodium bicarbonate between about 0.95% w/w and about 1.05% w/w of the solution; a percentage of glycerin between about 28.5% w/w and about 31.5% w/w of the solution; a percentage of sodium gluconate between about 0.38% w/w and about 0.42% w/w of the solution; a percentage of erythritol between about 3.8% w/w and about 4.2% w/w of the solution; a percentage of magnesium chloride between about 0.475% w/w and about 0.525% w/w of the solution; a percentage of zinc citrate between about 0.19% w/w and about 0.21% w/w of the solution; a percentage of potassium citrate between about 1.425% w/w and about 1.575% w/w of the solution; and a percentage of water between about 57% w/w and about 63% w/w of the solution. In a further aspect, the solution further comprises: pomegranate extract, wherein a percentage of pomegranate extract is between about 0.095% w/w and about 0.105% w/w of the solution; thyme extract, wherein a percentage of thyme extract is between about 0.095% w/w and about 0.105% w/w of the solution; licorice extract, wherein a percentage of licorice extract is between about 0.095% w/w and about 0.105% w/w of the solution, and; turmeric extract, wherein a percentage of turmeric extract is between about 0.095% w/w and about 0.105% w/w of the solution. In yet a further aspect, the solution further comprises: peppermint flavor extract, wherein a percentage of peppermint flavor extract is between about 0.19% w/w and about 0.21% w/w of the solution. In another aspect, the duration of mixing for mixing the water, zinc citrate, and potassium citrate is at least 1 hour. In yet another aspect, the duration of mixing the ingredients to produce a solution is at least 30 minutes.

### DETAILED DESCRIPTION OF THE INVENTION

In some aspects, an aqueous oral care composition (mouthwash) disclosed herein may comprise water. In another aspect, the aqueous oral care composition may comprise sodium bicarbonate. Sodium bicarbonate reduces tooth plaque formation and gingivitis.

In another aspect, the aqueous oral care composition may comprise glycerin. Glycerin is a natural preservative that prevents or reduces microbial growth. It can also improve the taste of the aqueous oral care composition, change the body, viscosity, and/or consistency of the aqueous oral care composition, and has moisturizing properties for the oral cavity, i.e., reduces dry mouth.

In another aspect, the aqueous oral care composition may comprise a sodium salt of an acid. In some aspects, the sodium salt is of an acid is selected from the group consisting of sodium gluconate and sodium benzoate. In yet a further aspect, the sodium salt of an acid is sodium gluconate. Sodium gluconate is a natural preservative that prevents or reduces microbial growth. It can also reduce bitterness and improve the taste of the mouthwash. Sodium gluconate also has moisturizing properties for the oral cavity, i.e., reduces dry mouth.

In another aspect, the aqueous oral care composition may comprise at least one sugar alcohol. In some aspects, the sugar alcohol is selected from the group consisting of erythritol, sorbitol, and xylitol. In a further aspect, the sugar alcohol is erythritol. Erythritol, a zero-calorie sweetener that promotes a healthy oral microbiome, supports tooth remineralization to repair enamel by forming complexes with calcium ions and possesses reduced cariogenic properties.

In another aspect, the aqueous oral care composition may comprise a chloride salt. In some aspects, the chloride salt is magnesium chloride, potassium chloride, calcium chloride, or sodium chloride. In a further aspect, the chloride salt is magnesium chloride. Magnesium chloride may help prevent or reduce tooth decay.

In another aspect, the aqueous oral care composition may comprise at least one citric acid salt. In some aspects the at least one citric acid salt is zinc citrate or potassium citrate. In a further aspect, the aqueous oral care composition comprises both zinc citrate and potassium citrate. Zinc citrate has antimicrobial and anti-inflammatory properties. Zinc citrate also prevents or reduces halitosis and formation of dental plaque and tartar, in part by preventing the formation of Streptococcus mutans biofilm. Potassium citrate reduces tooth pain and sensitivity. The combination of these citric acid salts is particularly advantageous for individuals who experience receding gums or sensitivity to temperature changes or sweetness.

In another aspect, the aqueous oral care composition may comprise at least one extract. In a further aspect, the aqueous oral care composition may comprise at least two extracts, alternatively at least three extracts, alternatively at least four extracts, alternatively at least five extracts. In yet another aspect, the extracts are selected from the group consisting of a flavor extract, an antimicrobial extract, an anti-inflammatory extract, an antioxidant extract, an anti-cavity extract, an antibacterial extract, an antiviral extract, an anti-inflammatory extract, a digestive extract, and an anti-allergy extract. In yet a further aspect, aqueous oral care composition may comprise pomegranate extract, thyme extract, licorice extract, turmeric extract, and peppermint extract, all of which impart beneficial properties for oral hygiene. Pomegranate extract has both antioxidant and antiviral properties, reduces inflammation of, and promotes healthy digestion. Thyme extract has antimicrobial, anti-inflammatory, and anti-allergy properties. Licorice extract possesses antibacterial and anti-cavity properties as well as reduces inflammation of the gut. Licorice extract also prevents, or reduces, halitosis in mouth and suppresses or reduces H. pylori gastritis in the gut. Turmeric extract has antioxidant and anti-inflammatory properties and promotes digestive health. Peppermint extract promotes healthy digestion and prevents, or reduces, halitosis.

In yet another aspect, the aqueous oral care composition comprises water, sodium bicarbonate, glycerin, sodium gluconate, erythritol, magnesium chloride, zinc citrate, potassium citrate, pomegranate extract, thyme extract, licorice extract, turmeric extract, and peppermint flavor extract.

In another aspect, the aqueous oral care composition may comprise sodium bicarbonate in the range of about 0.9% w/w to about 1.1% w/w of the formulation, alternatively in the range of about 0.95% w/w to about 1.05% w/w of the formulation. In another aspect, the aqueous oral care composition may comprise sodium gluconate in the range of about 0.36% w/w to about 0.44% w/w of the formulation, alternatively in the range of about 0.38% w/w to about 0.42% w/w of the formulation. In another aspect, the aqueous oral care composition may comprise glycerin in the range of about 27% w/w to about 33% w/w of the formulation, alternatively in the range of about 28.5% w/w to about 31.5% w/w of the formulation. In another aspect, the aqueous oral care composition may comprise erythritol in the range of about 3.6% w/w to about 4.4% w/w of the formulation, alternatively in the range of about 3.8% w/w to about 4.2% w/w of the formulation. In another aspect, the aqueous oral care composition may comprise magnesium chloride in the range of about 0.45% w/w to about 0.55% w/w of the formulation, alternatively in the range of about 0.475% w/w to about 0.525% w/w of the formulation. In another aspect, the aqueous oral care composition may comprise zinc citrate in the range of about 0.18% w/w to about 0.22% w/w of the formulation, alternatively in the range of about 0.19% w/w to about 0.21% w/w of the formulation. In another aspect, the aqueous oral care composition may comprise potassium citrate in the range of about 1.35% w/w to about 1.65% w/w of the formulation or in the range of about 1.425% w/w to about 1.575% w/w of the formulation. In another aspect, the aqueous oral care composition may comprise pomegranate extract in the range of about 0.09% w/w to about 0.11% w/w of the formulation, alternatively in the range of about 0.095% w/w to about 0.105% w/w of the formulation. In another aspect, the aqueous oral care composition may comprise thyme extract in the range of about 0.09% w/w to about 0.11% w/w of the formulation, alternatively in the range of about 0.095% w/w to about 0.105% w/w of the formulation. In another aspect, the aqueous oral care composition may comprise licorice extract in the range of about 0.09% w/w to about 0.11% w/w of the formulation, alternatively in the range of about 0.095% w/w to about 0.105% w/w of the formulation. In another aspect, the aqueous oral care composition may comprise turmeric extract in the range of about 0.09% w/w to about 0.11% w/w of the formulation, alternatively in the range of about 0.095% w/w to about 0.105% w/w of the formulation. In another aspect, the aqueous oral care composition may comprise peppermint extract in the range of about 0.18% w/w to about 0.22% w/w of the formulation or in the range of about 0.19% w/w to about 0.21% w/w of the formulation.

In some aspects of the aqueous oral care composition, water makes up the residual % w/w of the formulation. For example, water makes up about 54% w/w to about 66% w/w of the formulation or about 57% w/w to about 63% w/w of the formulation.

Some aspects of the aqueous oral care composition disclosed have a neutral pH. In another aspect, the aqueous oral care composition disclosed has a slightly basic pH. In yet another aspect, the pH of the aqueous oral care composition in the range of about 7 to about 9. A pH range of about 7 to about 9 provides naturally bacteriostatic or bactericidal properties without enamel erosion.

Some aspects of the aqueous oral care composition are ingestible. In further aspects, the aqueous oral care composition is alcohol-free. The alcohol-free nature of this aqueous oral care composition allows a user to ingest the aqueous oral care composition safely. In oral care compositions, alcohol is typically the preferred solvent, especially when dissolving extracts, including flavoring extracts such as peppermint or spearmint oils. Applicants have shown unexpectedly superior and surprising results, by being able to dissolve several extracts, including, but not limited to, pomegranate extract, thyme extract, licorice extract, turmeric extract, and peppermint flavor in an aqueous oral care composition. The incorporation of extracts in the aqueous oral care composition results in a pleasant sensation when the aqueous oral care composition is swallowed, further distinguishing the aqueous oral care composition from typical mouthwashes or other oral rise products.

Some aspects of the aqueous oral care composition disclosed are shelf-stable at room or ambient temperature. In another aspect of the aqueous oral care composition, the aqueous oral care composition is shelf-stable for at least one year, alternatively for at least two years, alternatively for at least three years.

In some aspects of the aqueous oral care composition, the aqueous oral care composition may comprise additional components including, but not limited to, fluoride ion sources, additional anti-tartar agents, buffers, abrasives, peroxide sources, alkali metal bicarbonate salts, thickening materials, humectants, surfactants, titanium oxide, coloring agents, additional flavor components, and mixtures thereof.

In another aspect of the aqueous oral care composition, abrasives to remove surface stains may include calcium carbonate, hydrated silica gels, hydrated aluminum oxides, magnesium carbonate, phosphate salts, and silicates.

In another aspect of the aqueous oral care composition, humectants can include glycerin such as vegetable glycerin and sorbitol.

In yet another aspect of the aqueous oral care composition, the aqueous oral care composition may include detergent or surfactant such as sodium lauryl sarcosinate, sodium coco sulphate, or sodium lauryl sulfate.

In yet another aspect of the aqueous oral care composition, the aqueous oral care composition may include mouthwash additives such as coconut oil extract, aloe barbadensis leaf extract, Irish moss, xanthum gum (or related high-molecular weight polysaccharide compounds), sea salt or derivatives thereof, phytocannabinoids such as cannabidiol (CBD), cannabinoids such as tetrahydrocannabinol (THC), or xylitol.

In further aspects, a method for making an aqueous oral care composition as disclosed herein comprising the steps of:
a. adding water to a vessel;
b. adding zinc citrate and potassium citrate to the vessel;
c. mixing the water, zinc citrate, and potassium citrate;
d. adding additional ingredients to the vessel including sodium bicarbonate, glycerin, sodium gluconate, erythritol, magnesium chloride, and at least one extract; and
e. mixing all of the ingredients into a solution.

In one aspect of the method, the extract may be one or more of a pomegranate extract, a thyme extract, a licorice extract, a turmeric extract, and/or a peppermint extract.

In another aspect of the method, the duration of mixing step “c” can be at least 1 hour, and the duration of mixing step “e” can be at least 30 minutes.

### EXAMPLES

### Mouthwash Formulation

Table 1 illustrates a non-acidic, alcohol-free, aqueous oral care composition according to some aspects of the present disclosure.

TABLE 1
Aqueous Oral Care Composition 1

| Ingredient | Percent kg |
|---|---|
| Water | 61.8 |
| Sodium Bicarbonate | 1.00 |
| Glycerin | 30.0 |
| Sodium Gluconate | 0.40 |
| Erythritol | 4.00 |
| Magnesium Chloride | 0.50 |
| Zinc Citrate | 0.20 |
| Potassium Citrate | 1.50 |
| Pomegranate Extract | 0.10 |
| Thyme Extract | 0.10 |
| Licorice Extract | 0.10 |
| Turmeric Extract | 0.10 |
| Peppermint Flavor | 0.20 |

61.8% w/w water was added to a mixing vessel. 0.20% w/w zinc citrate and 1.50% w/w potassium citrate were then added to the mixing vessel and mixed via a propeller mixer for an hour. The remaining ingredients of Table 1 were added to the mixing vessel and mixed for an additional 30 minutes until all ingredients were dissolved.

The presently described technology is now described in such full, clear, concise, and exact terms as to enable any person skilled in the art to which it pertains, to practice the same. It is to be understood that the foregoing describes preferred aspects of the technology and that modifications may be made therein without departing from the spirit or scope of the invention as set forth in the appended claims.

1. An aqueous oral care composition consisting of:
a. sodium bicarbonate;
b. glycerin;
c. sodium gluconate;
d. erythritol;
e. magnesium chloride;
f. zinc citrate;
g. potassium citrate;
h. water; and
i. a plurality of extracts.

2. (canceled)

3. The aqueous oral care composition of claim 1, wherein the plurality of extracts comprises a flavor extract, an antimicrobial extract, an anti-inflammatory extract, an antioxidant extract, an anti-cavity extract, an antibacterial extract, an antiviral extract, an anti-inflammatory extract, a digestive extract, and/or an anti-allergy extract.

4. The aqueous oral care composition of claim 3, wherein the plurality of extracts comprises a pomegranate extract, a thyme extract, a licorice extract, a turmeric extract, and/or a peppermint extract.

5. The aqueous oral care composition of claim 1, wherein the aqueous oral care composition has a pH in the range of 7 to 9.

6. The aqueous oral care composition of claim 1, wherein the aqueous oral care composition is ingestible.

7. The aqueous oral care composition of claim 1, wherein the aqueous oral care composition can be safely stored at room or ambient temperature for a usefully long shelf life.

8. The aqueous oral care composition of claim 7, wherein the aqueous oral care composition can be safely stored at room or ambient temperature for at least 1 year.

9. (canceled)

10. The aqueous oral care composition of claim 4, wherein the aqueous oral care composition formulation consists of:
a. a percentage of sodium bicarbonate between about 0.95% w/w and about 1.05% w/w of the formulation;
b. a percentage of glycerin between about 28.5% w/w and about 31.5% w/w of the formulation;
c. a percentage of sodium gluconate between about 0.38% w/w and about 0.42% w/w of the formulation;
d. a percentage of erythritol between about 3.8% w/w and about 4.2% w/w of the formulation;
e. a percentage of magnesium chloride between about 0.475% w/w and about 0.525% w/w of the formulation;
f. a percentage of zinc citrate between about 0.19% w/w and about 0.21% w/w of the formulation;
g. a percentage of potassium citrate between about 1.425% w/w and about 1.575% w/w of the formulation; and
h. a percentage of water between about 57% w/w and about 63% w/w of the formulation.

11. The aqueous oral care composition of claim 10, wherein the plurality of extracts of the aqueous oral care composition formulation consists of:
a. pomegranate extract, wherein a percentage of pomegranate extract is between about 0.095% w/w and about 0.105% w/w of the formulation;
b. thyme extract, wherein a percentage of thyme extract is between about 0.095% w/w and about 0.105% w/w of the formulation;
c. licorice extract, wherein a percentage of licorice extract is between about 0.095% w/w and about 0.105% w/w of the formulation;
d. turmeric extract, wherein a percentage of turmeric extract is between about 0.095% w/w and about 0.105% w/w of the formulation; and
e. peppermint flavor extract, wherein a percentage of peppermint flavor extract is between about 0.19% w/w and about 0.21% w/w of the formulation.

12. (canceled)

13. (canceled)

14. The aqueous oral care composition of claim 12, wherein the aqueous oral care composition is ingestible.

15. A method for producing an aqueous oral care composition comprising the steps of:
a. adding water to a vessel;
b. adding zinc citrate and potassium citrate to the vessel;
c. mixing the water, zinc citrate, and potassium citrate;
d. adding a plurality of additional ingredients to the vessel comprising sodium bicarbonate, glycerin, sodium gluconate, erythritol, magnesium chloride, and at least one extract; and
e. mixing the ingredients to produce a solution.

16. The method of claim 15, wherein the at least one extract is selected from the group of consisting of a flavor extract, an antimicrobial extract, an anti-inflammatory extract, an antioxidant extract, an anti-cavity extract, an antibacterial extract, an antiviral extract, an anti-inflammatory extract, a digestive extract, and an anti-allergy extract.

17. The method of claim 16, wherein the at least one extract is selected from the group consisting of a pomegranate extract, a thyme extract, a licorice extract, a turmeric extract, and a peppermint extract.

18. The method of claim 15, wherein the solution comprises:
a. a percentage of sodium bicarbonate between about 0.95% w/w and about 1.05% w/w of the solution;
b. a percentage of glycerin between about 28.5% w/w and about 31.5% w/w of the solution;
c. a percentage of sodium gluconate between about 0.38% w/w and about 0.42% w/w of the solution;
d. a percentage of erythritol between about 3.8% w/w and about 4.2% w/w of the solution;
e. a percentage of magnesium chloride between about 0.475% w/w and about 0.525% w/w of the solution;
f. a percentage of zinc citrate between about 0.19% w/w and about 0.21% w/w of the solution;
g. a percentage of potassium citrate between about 1.425% w/w and about 1.575% w/w of the solution; and
h. a percentage of water between about 57% w/w and about 63% w/w of the solution.

19. The method of claim 18, wherein the solution further comprises:
j. pomegranate extract, wherein a percentage of pomegranate extract is between about 0.095% w/w and about 0.105% w/w of the solution;
k. thyme extract, wherein a percentage of thyme extract is between about 0.095% w/w and about 0.105% w/w of the solution;
l. licorice extract, wherein a percentage of licorice extract is between about 0.095% w/w and about 0.105% w/w of the solution, and;
m. turmeric extract, wherein a percentage of turmeric extract is between about 0.095% w/w and about 0.105% w/w of the solution.

20. The method of claim 17, wherein the solution further comprises:
n. peppermint flavor extract, wherein a percentage of peppermint flavor extract is between about 0.19% w/w and about 0.21% w/w of the solution.

21. The method of claim 13, wherein the duration of mixing for step c is at least 1 hour.

22. The method of claim 13, wherein the duration of mixing for step e is at least 30 minutes.
